(19) United States
(12) Patent Application Publication
(10) Pub. No.: US 2019/0021680 A1
(43) Pub. Date: Jan. 24, 2019

(54) RADIATION IMAGING APPARATUS, RADIATION IMAGING SYSTEM, RADIATION IMAGING METHOD, AND COMPUTER-READABLE MEDIUM

(71) Applicant: CANON KABUSHIKI KAISHA, Tokyo (JP)

(72) Inventor: Jumpei Shirono, Yokohama-shi (JP)

(21) Appl. No.: 16/138,213

(22) Filed: Sep. 21, 2018

Related U.S. Application Data
(63) Continuation of application No. PCT/JP2017/003799, filed on Feb. 2, 2017.

(30) Foreign Application Priority Data
Mar. 29, 2016 (JP) 2016-066882

(51) Int. Cl. A61B 6/03; A61B 6/00; G06T 5/50; G01N 23/04

(52) U.S. Cl. CPC A61B 6/032; G01N 23/04; G06T 5/50; A61B 6/5282

(57) ABSTRACT

Provided is a radiation imaging apparatus including: an image acquisition unit configured to rotate a radiation generator and a radiation detector around a subject, to thereby acquire a plurality of radiographic images of the subject based on detected data on a radiation applied to the subject from a plurality of rotation angles; a first scattered radiation estimation unit configured to estimate a first scattered radiation distribution in a first radiographic image among the plurality of radiographic images; and a second scattered radiation estimation unit configured to use the first scattered radiation distribution to estimate a second scattered radiation distribution in a second radiographic image, which is different from the first radiographic image.

### CROSS-REFERENCE TO RELATED APPLICATIONS

This application is a Continuation of International Patent Application No. PCT/JP2017/003799, filed Feb. 2, 2017, which claims the benefit of Japanese Patent Application No. 2016-066882, filed Mar. 29, 2016, both of which are hereby incorporated by reference herein in their entirety.

### BACKGROUND OF THE INVENTION

### Field of the Invention

The present invention relates to a radiation imaging apparatus, a radiation imaging system, a radiation imaging method, and a computer-readable medium.

### Description of the Related Art

An X-ray CT apparatus developed in the 1970s have since been spreading and progressing. The X-ray CT apparatus includes an X-ray source (radiation generator) and a detector arranged so as to be opposed to the X-ray source across a subject, and measures X-rays while the X-ray source and the detector are rotated about the subject, to thereby measure X-rays transmitted through the subject at various angles. Then, an image of the subject is reconstructed through use of a filtered back projection (FBP) method or other such method based on information obtained from a result of the measurement. As a result, a spatial distribution of a linear attenuation coefficient of the subject may be obtained.

The CT apparatus measures to what extent a radiation applied from a radiation source (radiation generator) attenuates while traveling straight through the subject. As a result, the spatial distribution of the linear attenuation coefficient of the subject may be obtained. In this case, the measurement is performed under a state in which a radiation scattered by the subject is mixed, and hence accuracy is lowered in reconstruction of the spatial distribution of the linear attenuation coefficient. In view of this, it is required to estimate the mixed scattered radiation, and remove the mixed scattered radiation from a measured amount.

For example, in Japanese Patent No. 5388680, there is disclosed a method of speeding up scattered radiation estimation through use of a common scattered radiation distribution for “n” adjacent projection directions (see Japanese Patent No. 5388680).

However, in the method disclosed in Japanese Patent No. 5388680, the same scattered radiation distribution is used in the adjacent projection directions, which leads to a problem of poor accuracy.

### SUMMARY OF THE INVENTION

According to one embodiment of the present invention, there is provided a radiation imaging apparatus including: an image acquisition unit configured to rotate a radiation generator and a radiation detector around a subject, to thereby acquire a plurality of radiographic images of the subject based on detected data on the radiation applied to the subject from a plurality of rotation angles; a first scattered radiation estimation unit configured to estimate a first scattered radiation distribution in a first radiographic image among the plurality of radiographic images; and a second scattered radiation estimation unit configured to use the first scattered radiation distribution to estimate a second scattered radiation distribution in a second radiographic image, which is different from the first radiographic image.

Further features of the present invention will become apparent from the following description of exemplary embodiments with reference to the attached drawings.

### BRIEF DESCRIPTION OF THE DRAWINGS

FIG. 1 is a diagram for illustrating an example of a configuration of a radiation imaging system according to one embodiment of the present invention.

FIG. 2 is a flow chart for illustrating an example of a processing flow of a radiation imaging apparatus according to the one embodiment of the present invention.

FIG. 3 is a diagram for illustrating how a plurality of measured images are acquired in accordance with rotation of a rotation unit.

FIG. 4 is a graph for showing an example of a relationship between coordinates of a detector and an amount of scattered radiation.

FIG. 5 is a diagram for illustrating an example of a relationship between a resolution interval and a rotation angle.

### DESCRIPTION OF THE EMBODIMENTS

Preferred embodiments of the present invention will now be described in detail in accordance with the accompanying drawings.

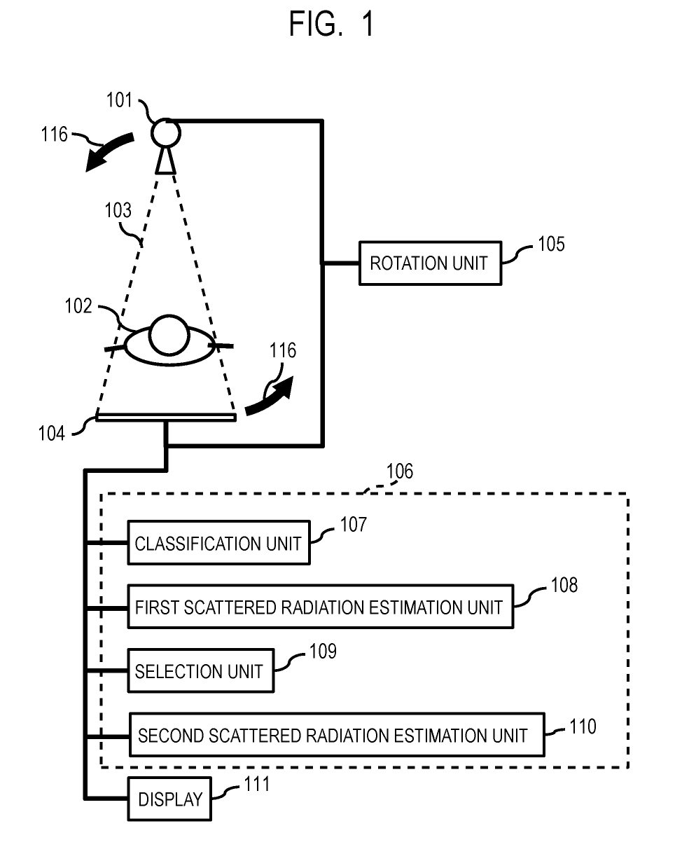

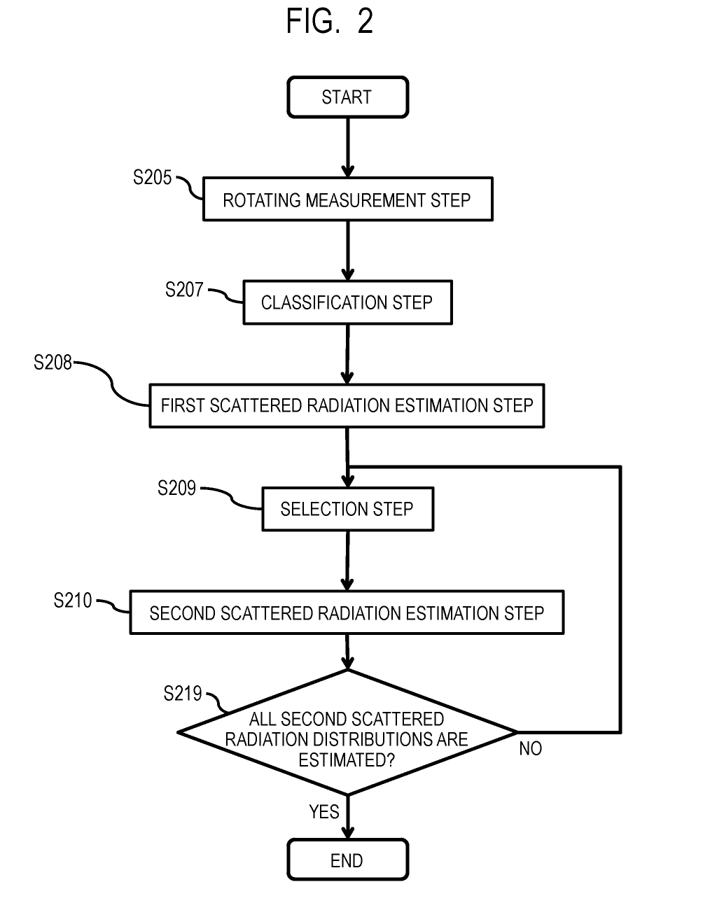

FIG. 1 is a diagram for illustrating an example of a configuration of a radiation imaging system according to one embodiment of the present invention. FIG. 2 is a flow chart for illustrating an example of a processing flow of a radiation imaging apparatus according to the one embodiment.

First, each of components illustrated in FIG. 1 is described. A radiation imaging system according to this embodiment includes a radiation generator (radiation generation source) 101, a detector 104, and a rotation unit 105. The radiation generator 101 is a radiation generation source, and applies a radiation 103 to a subject 102. In this embodiment, X-rays are used as a radiation, but alpha rays, beta rays, a heavy particle radiation, or gamma rays may be used.

In this embodiment, the subject 102 is a living body, but, for example, an industrial product may be used as the subject instead of the living body. The detector (radiation detector) 104 is arranged so as to be opposed to the radiation generator 101 across the subject 102, and detects the radiation 103 from the radiation generator 101.

In this embodiment, a flat panel detector (FPD) made from a semiconductor material so as to have a large number of detection elements arrayed in a grid is used as the detector 104, but a line sensor or other such detector may be used. In the FPD, the detection elements are arrayed two-dimensionally so as to allow measured information to be visualized like an image, and hence the measured information (detected data) of the detector 104 is referred to as “measured image” for the sake of convenience of description. The measured information of the line sensor or other such detector is also referred to as “measured image”.

The rotation unit 105 is a rotating measurement unit configured to rotate the radiation generator 101 and the detector 104 around the subject 102. In this embodiment, the radiation generator 101 and the detector 104 are rotated about the subject 102 in a rotation direction 116 in synchronization with each other, and the measurement is performed by the detector 104 while the subject 102 is exposed to the radiation 103 applied from the radiation generator 101, to thereby acquire a plurality of measured images at different rotation angles.

With a CT apparatus, the rotation unit 105 may be rotated in the rotation direction 116 by 360°, to thereby acquire a plurality of measured images at different rotation angles. With a C-arm apparatus, a plurality of measured images are acquired at different rotation angles in accordance with the rotation by less than 360°. With a tomosynthesis apparatus, the detector 104 is not rotated while the radiation generator 101 is rotated. The present invention can be applied to the radiation imaging apparatus of any kind.

The measured information obtained by the detector 104 is sent to an image processor 106 to be processed. The image processor 106 includes a classification unit 107, a first scattered radiation estimation unit 108, a selection unit 109, and a second scattered radiation estimation unit 110.

In this embodiment, the image processor 106 is a computer. In addition, the classification unit 107, the first scattered radiation estimation unit 108, the selection unit 109, and the second scattered radiation estimation unit 110 are functions that function based on programs executed by the computer. Each of the units is not required to be a function, and the form of an integrated circuit or other such form may be employed as long as the same function is achieved.

A display 111 displays a result obtained by each of the components in this embodiment. Examples of the display 111 include a liquid crystal display and a CRT. In addition, the display 111 may be any device that can be visually recognized by a human.

Next, with reference to the flow chart of FIG. 2, it is described what kind of operation is performed by each of the components of FIG. 1 to estimate a scattered radiation.

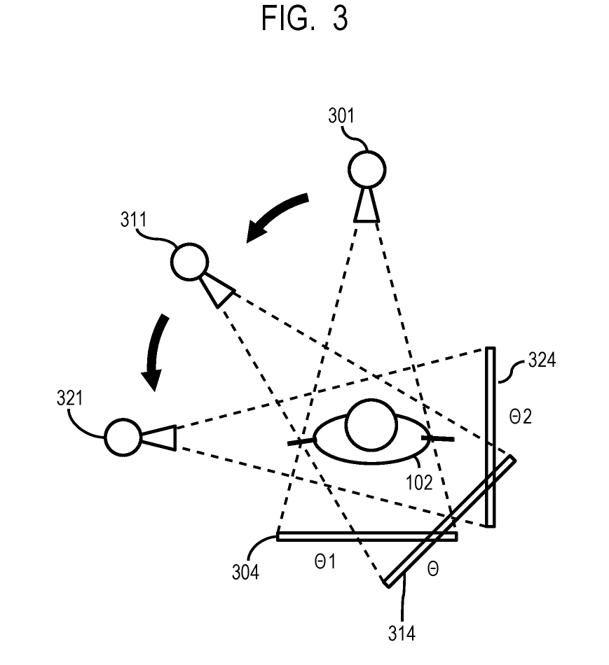

First, in Step S205, a rotating measurement step is executed. In this step, the rotation unit 105 is rotated, to thereby obtain a plurality of measured images at different rotation angles. FIG. 3 is a diagram for illustrating how a plurality of measured images are acquired in accordance with the rotation of the rotation unit 105. In FIG. 3, while the radiation generator 101 and the detector 104 are rotated to change the rotation angle, the subject 102 is exposed to the radiation 103 applied from each of positions 301, 311, and 321 of the radiation generator 101, and the radiation is detected at each of positions 304, 314, and 324 of the detector 104.

When the radiation generator 101 is located at the position 301, the detector 104 is located at the position 304. At the positions, the subject 102 is exposed to the radiation 103 applied from the radiation generation source, and the detector 104 measures the radiation 103 to obtain a measured image.

In order to obtain measured images at a different rotation angle, the rotation unit 105 is rotated to move the radiation generator 101 to the position 311 and move the detector 104 to the position 314. At the positions, the subject 102 is exposed to the radiation 103 applied from the radiation generation source, and the detector 104 measures the radiation 103 to obtain a measured image.

In order to obtain measured images at a further different rotation angle, the rotation unit 105 is rotated to move the radiation generator 101 to the position 321 and move the detector 104 to the position 324. At the positions, the subject 102 is exposed to the radiation 103 applied from the radiation generation source, and the detector 104 measures the radiation 103 to obtain a measured image.

In this manner, in this step, while the radiation generator 101 and the detector (radiation detector) 104 are rotated around the subject 102, the radiation 103 is applied to the subject 102 from a plurality of rotation angles. Then, an image acquisition unit (image processor 106) acquires a plurality of radiographic images of the subject 102 based on the detected data on the radiation 103.

With a CT apparatus, the rotation unit 105 may be rotated in the rotation direction 116 by 360°, to thereby acquire a plurality of measured images at different rotation angles. For the sake of description, a relatively large rotation angle is set in FIG. 3. However, in general, the rotation angle is set minute in order to enhance the resolution of the measured image of the subject 102. Further, in this embodiment, after the rotation unit 105 is rotated up to a predetermined rotation angle to be stopped, the subject 102 is exposed to the radiation 103, and the detector 104 obtains measured information. However, while the rotation unit 105 is moved without being stopped, the subject 102 may be exposed to the radiation 103, and the detector 104 may obtain measured information.

Subsequently, in Step S207, a classification step is executed. In this step, the classification unit 107 classifies the plurality of radiographic images measured (measured images obtained) in Step S205 into an image set A (first radiographic images) and an image set B (second radiographic images).

As illustrated in FIG. 3, in this embodiment, a measured image a₁ obtained when the detector 104 is located at the position 304 and a measured image a₂ obtained when the detector 104 is located at the position 324 are classified into the image set A (first radiographic images). A measured image b₁ obtained when the detector 104 is located at the position 314 is classified into the image set B (second radiographic images). It is described later on what criterion the measured images are classified into the image set A (first radiographic images) and the image set B (second radiographic images).

Subsequently, in Step S208, a first scattered radiation estimation step is executed. First scattered radiation distributions of the radiation 103 in the image set A (a plurality of first radiographic images) are estimated. In this step, the first scattered radiation estimation unit 108 estimates a scattered radiation distribution corresponding to each measured image of the image set A to obtain a scattered radiation distribution set Aₛ (first scattered radiation distributions). That is, the first scattered radiation estimation unit 108 estimates the first scattered radiation distributions in the first radiographic images among the plurality of radiographic images.

In this embodiment, scattered radiation distributions included in the measured image a₁ and the measured image a₂, which form the image set A, are estimated. A known method of estimating a scattered radiation distribution can be employed. As described in, for example, Japanese Patent No. 5052281, a path length by which a scattered radiation is transmitted through the subject 102, an absorption coefficient of the subject 102, and a scattering probability of the subject 102 are used to obtain a scattered radiation distribution.

The first scattered radiation estimation unit 108 obtains a scattered radiation distribution aₛ₁ corresponding to the measured image a₁ and a scattered radiation distribution aₛ₂ corresponding to the measured image a₂. Therefore, in this embodiment, the scattered radiation distribution set Aₛ includes the scattered radiation distribution aₛ₁ and the scattered radiation distribution aₛ₂.

Subsequently, in Step S209, a selection step is executed. In this step, the selection unit 109 selects one measured image “b” (second radiographic image) from the image set B. In this embodiment, the image set B is formed of the measured image b₁, and hence the measured image b₁ is selected.

Subsequently, in Step S210, a second scattered radiation estimation step is executed. The second scattered radiation estimation unit 110 uses the scattered radiation distribution set Aₛ (first scattered radiation distributions) to estimate a second scattered radiation distribution of the radiation 103 in the measured image “b” (second radiographic image). In this step, the second scattered radiation estimation unit 110 estimates a scattered radiation distribution “bₛ” (second scattered radiation distribution) in the measured image “b” from the scattered radiation distribution set Aₛ. That is, the second scattered radiation estimation unit 110 uses the first scattered radiation distributions to estimate the second scattered radiation distribution in the second radiographic image, which is different from the first radiographic image.

With reference to FIG. 3, this embodiment is described by taking an example of obtaining the scattered radiation distribution “bₛ” (second scattered radiation distribution) based on the scattered radiation distribution aₛ₁ (first scattered radiation distribution) and the scattered radiation distribution aₛ₂ (first scattered radiation distribution). As described above, in Step S205, measured images are obtained while the radiation generator 101 and the detector 104 are rotated. In accordance with the rotation of the rotation unit 105, the measured image is gradually changed, and the scattered radiation distribution in the measured image is also gradually changed. That is, as the detector 104 is rotationally moved to the positions 304, 314, and 324, the scattered radiation distribution is also gradually changed.

The scattered radiation distribution is gradually changed, and hence the scattered radiation distribution “bₛ” exhibited when the detector 104 is located at the position 314 can be obtained through interpolation from the scattered radiation distribution aₛ₁ exhibited when the detector 104 is located at the position 304 and the scattered radiation distribution aₛ₂ exhibited when the detector 104 is located at the position 324. For example, the scattered radiation distribution “bₛ” is obtained by Expression (1).

$${b}_{s} ( {ξ}_{i} ) = \frac{1}{{θ}_{2} - {θ}_{1}} \{ ( {θ}_{2} - θ ) {a}_{s 1} ( {ξ}_{i} ) + ( θ - {θ}_{1} ) {a}_{s 2} ( {ξ}_{i} ) \} \qquad ( 1 )$$

In Expression (1), ξᵢ represents the value of a scattered radiation distribution at an i-th pixel of the FPD. Further, θ₁ represents a rotation angle exhibited when the detector 104 is located at the position 304, θ2 represents a rotation angle exhibited when the detector 104 is located at the position 324, and “θ” represents a rotation angle exhibited when the detector 104 is located at the position 314. That is, the value of the scattered radiation distribution “bₛ” (second scattered radiation distribution) at a predetermined pixel of the measured image “b” (second radiographic image) is estimated by being interpolated from the values of the scattered radiation distributions “aₛ” (first scattered radiation distributions) at the same pixel of a plurality of measured images “a” (first radiographic images) through use of the rotation angle.

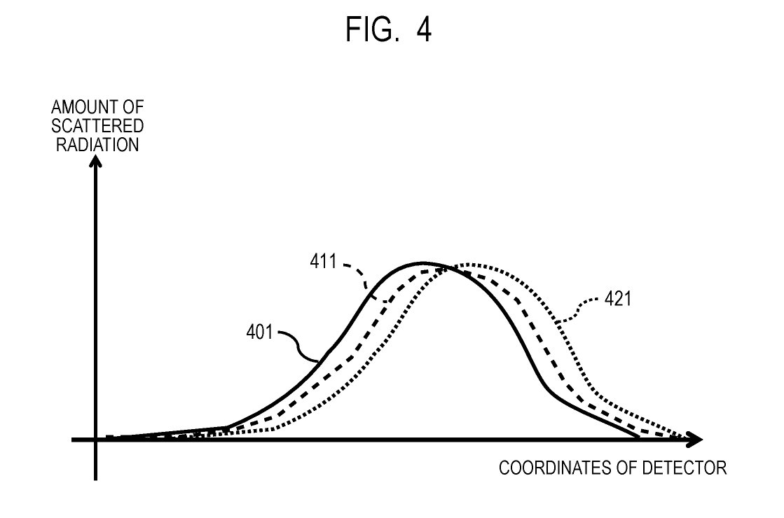

FIG. 4 is a graph for showing an example of a relationship between coordinates of the detector 104 and an amount of scattered radiation that are exhibited when “θ” is present within an acute angle defined by the rotation angles θ₁ and θ₂. In FIG. 4, the horizontal axis indicates the coordinates of the detector 104, and the vertical axis indicates the amount of scattered radiation. In FIG. 4, a scattered radiation distribution 401 is the scattered radiation distribution aₛ₁ exhibited when the detector 104 is located at the position 304. A scattered radiation distribution 421 is the scattered radiation distribution aₛ₂ exhibited when the detector 104 is located at the position 324. A scattered radiation distribution 411 is the scattered radiation distribution “bₛ” exhibited when the detector 104 is located at the position 314, which is the scattered radiation distribution “bₛ” obtained through the interpolation.

The classification unit 107 classifies a radiographic image acquired within the acute angle defined by the rotation angles θ₁ and θ₂ for the plurality of measured images “a” (first radiographic images) as the measured image “b” (second radiographic image). The scattered radiation distribution “bₛ” in the measured image “b” (second radiographic image) is estimated by being interpolated from the values of the scattered radiation distributions aₛ₁ and aₛ₂ at the same pixel of the plurality of measured images “a” (first radiographic images) through use of the rotation angles θ₁, θ₂, and “θ”.

In general, the resolution of a scattered radiation distribution required for correction is often lower than in the measured image to be obtained. Therefore, the method of estimating a scattered radiation involving a large calculation amount, which is used in Step S208, is not always required to be executed on all the measured images obtained through rotating measurement, and it is possible to obtain a sufficiently accurate scattered radiation distribution with a small calculation amount through the interpolation performed from the scattered radiation distributions obtained in Step S208.

Further, the second scattered radiation distribution of the image set B (second radiographic images) is estimated from the first scattered radiation distributions of the image set A (first radiographic images), and hence the radiographic image can be classified so as to facilitate the estimation (Step S207). As described above, when a scattered radiation distribution is interpolated based on the rotation angle, of the three radiographic images acquired consecutively, the first and last radiographic images can be classified into the image set A, and the intermediate radiographic image can be classified into the image set B.

The classification unit 107 may classify a freely-selected radiographic image into the image set B (second radiographic images), and may classify two radiographic images acquired at the closest rotation angles before and after a rotation angle for the image set B (second radiographic images) into the image set A (first radiographic images).

In this case, the first scattered radiation estimation unit 108 estimates the first scattered radiation distributions aₛ₁ and aₛ₂ in the two first radiographic images a₁ and a₂, respectively. The second scattered radiation estimation unit 110 performs the interpolation from the first scattered radiation distributions aₛ₁ and aₛ₂ in the two first radiographic images a₁ and a₂ based on the rotation angles θ₁ and θ₂ for the two first radiographic images a₁ and a₂ and the rotation angle “θ” for the second radiographic image “b”. The second scattered radiation estimation unit 110 estimates the second scattered radiation distribution “bₛ” in the second radiographic image “b” through the interpolation.

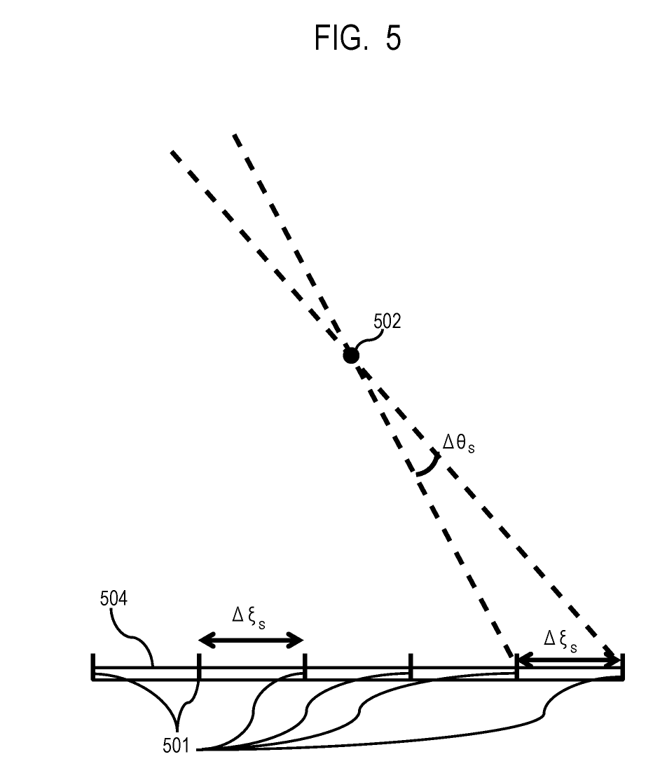

An interval between each pair of rotation angles for images of the image set A can be suppressed to, for example, about Δθₛ of FIG. 5 (or within a predetermined rotation angle). In FIG. 5, sampling points 501 of the scattered radiation distribution on a detector 504 are arranged at intervals of Δξₛ. In other words, Δξₛ represents a resolution interval of the scattered radiation distribution.

Δθₛ is a rotation angle of the rotation unit 105 corresponding to the resolution interval Δξₛ. When the rotation unit 105 is rotated about the rotation center 502 by the rotation angle Δθₛ, a moving distance of a detection surface of the detector 504 corresponds to the resolution interval Δξₛ. In FIG. 5, the rotation angle Δθₛ is smaller at an end portion of the detection surface than at a central portion of the detection surface, to thereby obtain the rotation angle Δθₛ through use of the resolution interval Δξₛ at the end portion of the detection surface of the detector 504.

When Δξₛ is sufficiently smaller than a rotation radius R of the rotation unit 105, the rotation angle Δθₛ may be obtained by Expression (2).

Δθₛ=tan⁻¹(Δξₛ/R) (2)

In order to suppress the interval between each pair of the rotation angles for the images of the image set A to about Δθₛ, the classification unit 107 classifies a radiographic image acquired at the rotation angle Δθₛ corresponding to the resolution interval Δξₛ of the first scattered radiation distributions or acquired within the rotation angle Δθₛ into the image set A (first radiographic images).

This embodiment is described by taking an example of linear interpolation with reference to Expression (1), but the kind of the interpolation is not limited thereto. For example, with quadratic interpolation, three images may be selected from the scattered radiation distribution set Aₛ of the first scattered radiation distributions to perform interpolation processing. It is also possible to use extrapolation to estimate the second scattered radiation distribution. The second scattered radiation estimation unit 110 estimates the second scattered radiation distribution by performing interpolation or extrapolation on a plurality of first scattered radiation distributions.

Subsequently, in Step S219, the selection unit 109 determines whether or not all the second scattered radiation distributions have been estimated for the image set B. When not all the second scattered radiation distributions have been estimated, the processing returns to Step S209, and the selection step and the second scattered radiation estimation step are further executed. Meanwhile, when all the second scattered radiation distributions have been estimated, the processing for estimating a scattered radiation distribution is brought to an end.

In the above-mentioned manner, in Steps S209, S210, and S219, all the second scattered radiation distributions corresponding to the second radiographic images of the image set B are estimated.

As described above, the first scattered radiation distribution is estimated for the first radiographic image of the image set A in Step S208, and the second scattered radiation distribution is estimated for the second radiographic image of the image set B in Step S210. Through this estimation, the scattered radiation distributions are estimated for all the measured images including the first radiographic image and the second radiographic image.

Further, the display 111 may display the obtained scattered radiation distribution as required. This allows the accuracy of the scattered radiation estimation to be confirmed.

According to this embodiment, a scattered radiation can be estimated with high accuracy. In particular, by performing the interpolation processing through use of the rotation angle, it is possible to estimate a scattered radiation with higher accuracy than by the method using the same scattered radiation distribution in adjacent projection directions, which is described in Japanese Patent No. 5388680. Further, according to this embodiment, a scattered radiation is estimated by the interpolation processing, and hence it is possible to obtain a sufficiently accurate scattered radiation distribution with a small calculation amount.

With the radiation imaging apparatus according to the embodiment, it is possible to estimate a scattered radiation with high accuracy.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions

What is claimed is:

1. A radiation imaging apparatus comprising:
an image acquisition unit configured to rotate a radiation generator and a radiation detector around a subject, to thereby acquire a plurality of radiographic images of the subject based on detected data on a radiation applied to the subject from a plurality of rotation angles;
a first scattered radiation estimation unit configured to estimate a first scattered radiation distribution in a first radiographic image among the plurality of radiographic images; and
a second scattered radiation estimation unit configured to use the first scattered radiation distribution to estimate a second scattered radiation distribution in a second radiographic image, which is different from the first radiographic image.

2. A radiation imaging apparatus according to claim 1, further comprising a classification unit configured to classify the plurality of radiographic images into the first radiographic image and the second radiographic image.

3. A radiation imaging apparatus according to claim 2, wherein the classification unit is configured to classify a radiographic image acquired at an interval between each pair of the plurality of rotation angles, which corresponds to a resolution interval of the first scattered radiation distribution, as the first radiographic image.

4. A radiation imaging apparatus according to claim 2,
wherein the first radiographic image includes a plurality of first radiographic images, and
wherein the classification unit is configured to classify a radiographic image acquired within an acute angle defined by one pair of the plurality of rotation angles for the plurality of first radiographic images, as the second radiographic image.

5. A radiation imaging apparatus according to claim 1,
wherein the first radiographic image includes a plurality of first radiographic images, and
wherein the second scattered radiation estimation unit is configured to estimate the second scattered radiation distribution through use of the first scattered radiation distribution in each of the plurality of first radiographic images acquired at one pair of the plurality of rotation angles including therebetween one of the plurality of rotation angles that is used when the second radiographic image is acquired.

6. A radiation imaging apparatus according to claim 1,
wherein the first scattered radiation distribution includes a plurality of first scattered radiation distributions, and
wherein the second scattered radiation estimation unit is configured to perform one of interpolation and extrapolation on the plurality of first scattered radiation distribution, to thereby estimate the second scattered radiation distribution.

7. A radiation imaging system comprising:
a radiation generator configured to apply a radiation to a subject;
a radiation detector, which is arranged so as to be opposed to the radiation generator across the subject, and is configured to detect the radiation applied from the radiation generator;
a rotation unit configured to rotate the radiation generator and the radiation detector around the subject;
an image acquisition unit configured to acquire a plurality of radiographic images of the subject based on detected data on the radiation applied to the subject from a plurality of rotation angles by rotating the radiation generator and the radiation detector around the subject;
a first scattered radiation estimation unit configured to estimate a first scattered radiation distribution in a first radiographic image among the plurality of radiographic images; and
a second scattered radiation estimation unit configured to use the first scattered radiation distribution to estimate a second scattered radiation distribution in a second radiographic image, which is different from the first radiographic image.

8. A radiation imaging method comprising:
rotating a radiation generator and a radiation detector around a subject, to thereby acquire a plurality of radiographic images of the subject based on detected data on a radiation applied to the subject from a plurality of rotation angles;
estimating a first scattered radiation distribution in a first radiographic image among the plurality of radiographic images; and
using the first scattered radiation distribution to estimate a second scattered radiation distribution in a second radiographic image, which is different from the first radiographic image.

9. A non-transitory computer-readable medium having stored thereon a program for causing, when executed by a computer, the computer to execute each step of the radiation imaging method of claim 8.
